(19) United States
(12) Patent Application Publication
(10) Pub. No.: US 2007/0036848 A1
(43) Pub. Date: Feb. 15, 2007

(54) Estrogen compositions and therapeutic methods of use thereof

(75) Inventors: Jonathan Bortz, St. Louis, MO (US); Thomas C. Riley JR., St. Charles, MO (US)

Correspondence Address: HARNESS, DICKEY, & PIERCE, P.L.C, 7700 BONHOMME, STE 400, ST. LOUIS, MO, 63105, US

(73) Assignee: DrugTech Corporation, Wilmington, DE (US)

(21) Appl. No.: 11/502,253

(22) Filed: Aug. 10, 2006

Related U.S. Application Data
(60) Provisional application No. 60/707,662, filed on Aug. 12, 2005.

(51) Int. Cl. A61K 31/56; A61K 9/127

(52) U.S. Cl. 424/450; 514/182

(57) ABSTRACT

A pharmaceutical composition comprises at least one estrogenic compound, the composition being adapted for application in a unit dose amount to a vulvovaginal surface and having at least one nonlipoidal internal phase and at least one lipoidal external phase that is bioadhesive to the vulvovaginal surface, wherein the at least one estrogenic compound is present in an amount of about 5 to about 1000 μg estradiol equivalent per unit dose of the composition, and upon application of the composition to the vulvovaginal surface the at least one estrogenic compound is released over a period of about 3 hours to about 30 days. The composition is useful for vulvovaginal administration to treat atrophic vaginitis or a disorder associated therewith, for example in a menopausal or postmenopausal woman. A method for treating a hypoestrogenism-related condition of the urogenital system of a female patient comprises intravaginal administration of at least one estrogenic compound according to a treatment regimen wherein a series of compositions releasing a progressively increasing daily amount of the at least one estrogenic compound is administered over a period of at least about 1 month.

This application claims the benefit of U.S. provisional patent application Ser. No. 60/707,662, filed on Aug. 12, 2005, the entire disclosure of which is incorporated by reference herein. This application contains subject matter that is related to concurrently filed U.S. application Ser. No. ______, titled “Therapeutic methods of using estrogen compositions”, the entire disclosure of which is incorporated by reference herein.

### FIELD OF THE INVENTION

The present invention relates to pharmaceutical compositions suitable for vaginal delivery of an estrogen compound. The invention further relates to therapeutic methods of use of such compositions in women having conditions of the urogenital system that are related to diminished levels of estrogen, as occur during and after menopause.

### BACKGROUND OF THE INVENTION

A major problem for menopausal and postmenopausal women is that the diminished supply of natural estrogen accompanying menopause leads to a variety of disorders, including disorders of the urogenital system. Such disorders can be ameliorated or corrected by administration of estrogenic compounds such as estradiol, ethinyl estradiol, conjugated estrogenic hormones, estriol and/or estrone, either locally (e.g., intravaginally) or systemically (e.g., orally or transdermally).

Among such disorders are urogenital atrophic disorders, for example atrophic vaginitis, a disorder characterized by dryness, soreness, pruritus and/or irritation of the vagina and/or vulva, and loss of elasticity of the vaginal wall. Secondarily, these conditions can lead to dyspareunia, which makes sexual activity uncomfortable or painful, and to urinary incontinence and/or increased incidence of urinary tract infections.

Atrophic vaginitis associated with postmenopausal hypoestrogenism is readily treatable with estrogenic compounds, including by vaginal administration. Safety and efficacy for vaginal atrophy of vaginal estrogen preparations have been reviewed by Crandall (2002), Journal of Women's Health 11(10):857-877.

However, even local vaginal administration of estrogenic compounds can give rise to significantly increased systemic levels of estrogen, which have been associated with adverse effects including endometrial hyperplasia, and which some studies have suggested can lead to a higher risk of breast cancer, more specifically higher risk of recurrence of breast cancer in breast cancer survivors. It is therefore desired to efficiently deliver estrogenic compounds locally to the urogenital system while minimizing systemic delivery, and a need exists for improved compositions and methods of use thereof to achieve this.

Rioux et al. (2000), Menopause 7(3):156-161, reported that treatment of atrophic vaginitis with estradiol (Vagifem®, Novo Nordisk) vaginal tablets led to reduced frequency of patients having systemic estradiol concentrations outside the normal postmenopausal range (i.e., >49 pg/ml) by comparison with conjugated equine estrogen (Premarin®, Wyeth-Ayerst) vaginal cream. They attributed the difference in part to the smaller estrogen dose in the vaginal tablets (25 μg versus 1.25 mg) and to the slow-release formulation of the estradiol in the vaginal tablets.

U.S. Pat. No. 4,551,148 to Riley et al. proposes a controlled release system for vaginal drug delivery, comprising unit cells having a nonlipoidal internal phase and a lipoidal continuous external phase. An active agent is present at least in the internal phase

U.S. Pat. No. 5,266,329 to Riley proposes such a vaginal delivery system having an antifungal as the active agent.

Thompson & Levinson (2002), Drug Delivery Systems & Sciences 2(1), 17-19, describe the VagiSite® bioadhesive topical drug delivery system as a high internal phase ratio water-in-oil emulsion system, providing a delivery platform for administration of active drug entities in the vaginal cavity. They disclose that the VagiSite® system is incorporated in Gynazole-1® antifungal vaginal cream.

U.S. Patent Application Publication No. 2003/0180366 of Kirschner et al. proposes a vaginal drug delivery system having globules comprising an internal water-soluble phase that is acid buffered and contains a drug, and an external water-insoluble phase or film.

U.S. Patent Application Publication No. 2004/0234606 of Levine et al. proposes a composition for vaginal administration comprising a treating agent (the tocolytic drug terbutaline is exemplified) and a bioadhesive cross-linked water-swellable but water-insoluble polycarboxylic acid such as polycarbophil, designed to give controlled and prolonged release of the drug through the vaginal mucosa. Administration of the composition is said to achieve local tissue concentrations without detrimental blood levels.

### SUMMARY OF THE INVENTION

There is now provided a pharmaceutical composition comprising at least one estrogenic compound, the composition being adapted for application in a unit dose amount to a vulvovaginal surface, for example a vaginal mucosal surface. The composition has at least one nonlipoidal internal phase and at least one lipoidal external phase that is bioadhesive to the vulvovaginal surface. The at least one estrogenic compound is present in an amount of about 5 to about 1000 μg estradiol equivalent per unit dose of the composition, and upon application of the composition to the vulvovaginal surface the at least one estrogenic compound is released over a period of about 3 hours to about 30 days, more particularly about 2 to about 14 days.

In a particular embodiment the composition contains the at least one estrogenic compound in an amount of about 5 to about 500 μg estradiol equivalent per unit dose, and is adapted for slow release thereof, for example over a period of about 2 to about 14 days.

The composition is typically a water-in-oil emulsion of a type described in the pharmaceutical art as a cream.

There is further provided a vaginal estrogen delivery system comprising such a cream and an applicator to facilitate administration to a vaginal mucosal surface.

There is still further provided a method for treating a hypoestrogenism-related condition of the urogenital system of a female patient, the method comprising administration to a vulvovaginal surface, for example a vaginal mucosal surface, of a pharmaceutical composition as described herein comprising about 5 to about 500 μg estradiol equivalent per unit dose.

There is still further provided a method for treating a hypoestrogenism-related condition of the urogenital system of a female patient, the method comprising intravaginal administration of at least one estrogenic compound according to a treatment regimen wherein a series of compositions releasing a progressively increasing daily amount of the at least one estrogenic compound is administered over a period of at least about 1 month.

Additional embodiments are described in the detailed description that follows.

### DETAILED DESCRIPTION

A composition of the invention can illustratively take the form of a water-in-oil emulsion as generally described in any of above-referenced U.S. Pat. Nos. 4,551,148, 5,266,329 or U.S. Patent Application Publication No. 2003/0180366, or as further described in U.S. Patent Application Publication No. 2005/0095245 of Riley et al., but differs from these at least in that it comprises an estrogenic compound as active agent. Such a water-in-oil emulsion can be presented in a semi-solid form, for example as a vaginal cream. As indicated above, the estrogenic compound is present in an amount of about 5 to about 1000 μg, for example about 5 to about 500 μg, estradiol equivalent per unit dose of the composition. Furthermore, the composition is formulated to release the estrogenic compound over a period of about 3 hours to about 30 days, for example about 3 hours to about 14 days, or about 3 hours to about 10 days, when applied to a vulvovaginal surface.

A “vulvovaginal surface” herein denotes any external or internal surface of the female genitalia, including mucosal surfaces in the vaginal cavity and nonmucosal surfaces of the vulva and immediately surrounding areas of skin. In some embodiments, the composition is more specifically adapted for application to a vaginal mucosal surface, and the external phase of the composition is bioadhesive to such a surface.

In one embodiment, the composition is formulated as the VagiSite® bioadhesive topical drug delivery system described by Thompson & Levinson (2002), op. cit., or a delivery system substantially equivalent thereto, with inclusion of at least one estrogenic compound as active agent.

An “estrogenic compound” herein is any compound or mixture thereof, whether of natural, biosynthetic or chemosynthetic origin, having estrogenic activity in a human female. Estrogenic compounds include steroidal and nonsteroidal compounds. Illustrative nonsteroidal estrogenic compounds include without limitation broparoestrol, chlorotrianisene, dienestrol, diethylstilbestrol, fosfestrol, hexestrol, methestrol, derivatives such as salts and esters thereof, enantiomers and racemates thereof, mixtures thereof and the like. Illustrative steroidal estrogenic compounds include without limitation conjugated estrogenic hormones (e.g., Premarin®), equilenin, equilin, estradiol, estriol, estrone, ethinyl estradiol, mestranol, moxestrol, quinestradiol, quinestrol, derivatives such as salts and esters thereof, enantiomers and racemates thereof, mixtures thereof and the like.

In an illustrative embodiment the at least one estrogenic compound comprises a steroidal compound.

In a more particular illustrative embodiment the at least one estrogenic compound comprises a compound selected from the group consisting of conjugated estrogenic hormones, estradiol, ethinyl estradiol, estriol and estrone.

In a still more particular illustrative embodiment the at least one estrogenic compound comprises estradiol or a derivative thereof, e.g., ethinyl estradiol.

Amounts of the at least one estrogenic compound are expressed herein as estradiol equivalent amounts unless the context demands otherwise. A composition of the invention provides about 5 to about 1000 μg, for example about 10 to about 500 μg, estradiol equivalent per unit dose. In various embodiments the at least one estrogenic compound is present in a total estradiol equivalent amount of about 20 to about 450 μg, about 25 to about 400 μg, about 25 to about 250 μg, about 25 to about 150 μg or, illustratively, about 25 μg, about 50 μg, about 100 μg, about 150 μg, about 200 μg, about 250 μg, about 300 μg, about 350 μg or about 400 μg, per unit dose of the composition.

A unit dose is an amount of the composition suitable for a single administration to a vulvovaginal surface, for example a vaginal mucosal surface, as described herein. Most conveniently for the patient, the composition is provided in unit dose aliquots, typically individually packaged, but this is not a requirement of the present invention. A convenient unit dose aliquot of a vaginal cream is an amount of about 1 to about 10 g, although greater or lesser amounts, for example as little as about 0.1 g or as much as about 25 g, or about 0.2 to about 10 g, about 0.25 to about 5 g or about 0.5 to about 2 g, can be used if desired. A particularly suitable unit dosage amount of a vaginal cream is about 2 to about 6 g, for example about 2 g, about 3 g, about 4 g or about 5 g. Where a unit dose is an amount of about 1 g, the total estradiol equivalent concentration in the composition is about 5 to about 1000 μg/g, for example about 10 to about 500 μg/g; in various embodiments about 20 to about 450 μg/g, about 25 to about 400 μg/g, about 25 to about 250 μg/g or about 25 to about 150 μg/g. Where the unit dose is greater or smaller than 1 g, suitable estradiol equivalent concentration ranges will be correspondingly lower or higher respectively. For example, where a unit dose is an amount of about 5 g, the total estradiol equivalent concentration in the composition is about 1 to about 200 μg/g, for example about 2 to about 100 pg/g; in various embodiments about 4 to about 90 μg/g, about 5 to about 80 μg/g, about 5 to about 50 μg/g or about 5 to about 30 μg/g.

In one particular low-dose embodiment, the total estradiol equivalent concentration of the composition is about 5 to about 250 μg/g, for example about 10 to about 150 μg/g or about 20 to about 100 μg/g. For some uses, a cream having an estradiol equivalent concentration substantially lower than about 100 μg/g (about 0.01%), for example about 20, about 25, about 40, about 50, about 60 or about 75 μg/g, can be provided. In some situations, the estradiol equivalent concentration of such a cream can be less than about 50 μg/g (about 0.005%), for example less than about 25, less than about 15 or less than about 5 μg/g.

In another particular low-dose embodiment, the total estradiol equivalent concentration, release rate (as more fully described hereinbelow) and unit dose are such as to provide delivery of about 2 to about 75 μg, for example about 5 to about 50 μg, illustratively about 7, about 14, about 21, about 28 or about 42 μg, estradiol equivalent per day.

Conveniently, a unit dosage amount of a vaginal cream of the invention can be furnished in a prefilled container or applicator, for example an applicator similar to that used for Gynazole-1® vaginal cream of KV Pharmaceutical Co., St Louis, Mo.

An estrogen delivery system comprising a vaginal cream composition of the invention, for example a disposable applicator, more particularly a disposable applicator prefilled with a unit dose of the composition, is an embodiment of the invention.

The at least one estrogenic compound can be present in either one or both of the internal and external phases. In one embodiment the at least one estrogenic compound is present at least in part in the internal phase of the composition, and can be in dispersed form, for example in solution or suspension therein, or in non-dispersed form. Optionally, substantially all of the at least one estrogenic compound can be present in the internal phase. Solubilization of the at least one estrogenic compound can be achieved, for example, by use of a cosolvent and/or surfactant. The at least one estrogenic compound can be present at least in part in particulate form, for example in micronized form, and can be dispersed as a particulate suspension in the internal and/or external phase. In various embodiments the at least one estrogenic compound is present in solution, in aggregates, in liposomes, in microcapsules and/or in micelles within the internal and/or external phase. If present in both internal (nonlipoidal) and external (lipoidal) phases, the at least one estrogenic compound can be present in similar or different amounts in the nonlipoidal and lipoidal phases.

The composition is adapted to release the at least one estrogenic compound over a period of about 3 hours to about 30 days, upon application to a vulvovaginal surface, for example a vaginal mucosal surface. Based on the disclosure herein, including disclosure of documents incorporated by reference herein, in particular above-referenced U.S. Pat. Nos. 4,551,148 and 5,266,329, and U.S. Patent Application Publication Nos. 2003/0180366 and 2005/0095245, one of skill in the art can without undue experimentation adjust release rate of the at least one estrogenic compound from the composition to achieve a release period of about 3 hours to about 30 days as required herein. In one embodiment, the release period is one of about 12 hours to about 10 days, for example about 1 to about 10 days, about 2 to about 10 days or about 3 to about 7 days. In other embodiments, the release period is one of about 3 hours to about 14 days, about 12 hours to about 14 days, about 1 to about 14 days, about 3 to about 14 days or about 15 to about 30 days. In particular embodiments, the release period is such that the composition is adapted for once daily to once monthly administration, for example about 1 to about 2 times per month or about 1 to about 3 times per week.

Release rate can be determined by in vivo testing or by any suitable in vitro method. An illustrative in vitro method utilizes an open chamber diffusion cell system such as a Franz cell system, typically fitted with an appropriate inert synthetic membrane such as polysulfone, cellulose acetate/nitrate mixed ester or polytetrafluoroethylene of suitable thickness, e.g., 70 μm. The receptor medium should be one in which the estrogenic compound of interest is soluble, for example a water/ethanol medium. A test composition is placed uniformly on the membrane (illustratively, about 300 mg of a semi-solid composition such as a cream is a suitable amount for placement on a 25 mm diameter membrane) and is kept occluded to prevent solvent evaporation and compositional changes. This corresponds to an infinite dose condition. An aliquot of the receptor fluid is removed for analysis at appropriate intervals, and is replaced with an aliquot of fresh receptor fluid, so that the membrane remains in contact with the receptor fluid throughout the period of the release study. A release rate study such as that outlined above is typically replicated and can be conducted using a standard composition having known release properties for comparison.

A “release period” or equivalent phrase herein refers to a period during which the at least one estrogenic compound is made available for absorption and pharmacological effect at or close to the site of absorption, for example the vaginal cavity, in an amount sufficient to provide therapeutic benefit or prophylaxis with respect to a hypoestrogenism-related local condition, for example atrophic vaginitis.

The composition typically comprises unit cells each having internal and external phases. The at least one internal phase can be discontinuous and is nonlipoidal and generally miscible with water. Illustratively, the internal phase comprises water, glycerin, propylene glycol, sorbitol or a combination of two or more thereof. The internal phase can itself be monophasic, biphasic or multiphasic, taking the form for example of a solution, suspension, emulsion or combination thereof. The internal phase can comprise one or more suspended solids, osmotic agents, extenders, diluents, buffers, chelating agents, preservatives or other materials. Optionally, the internal phase is acid buffered to an internal pH of about 2.0 to about 6.0, for example about 2.5 to about 5.5 or about 3.5 to about 5.0. In one embodiment the internal phase is acid buffered to an internal pH that is substantially optimal to the vaginal environment, i.e., a pH that does not cause substantial irritation, itching or other discomfort and/or is detrimental to common pathogens of the vaginal cavity, including fungal pathogens such as Candida species and bacterial pathogens such as Enterococcus species. Typically such a pH is approximately 4.5.

The external phase is lipoidal and generally continuous. The term “lipoidal” herein can pertain to any of a group of organic compounds including neutral fats, fatty acids, waxes, phosphatides, petrolatum, fatty acid esters of monoprotic alcohols, mineral oils, etc., having the following properties: insoluble in water; soluble in alcohol, ether, chloroform or other fat solvents; and exhibiting a greasy feel. Examples of suitable oils are mineral oils having viscosity of about 5.6 to about 68.7 centistokes, for example about 25 to about 65 centistokes, and vegetable oils such as coconut, palm kernel, cocoa butter, cottonseed, peanut, olive, palm, sunflower, sesame, corn, safflower, rapeseed (canola) and soybean oils and fractionated liquid triglycerides of naturally derived short-chain fatty acids.

The term “lipoidal” can also pertain to amphiphilic compounds, including for example natural and synthetic phospholipids. Suitable phospholipids can include, for example phosphatidylcholine esters such as dioleoylphosphatidylcholine, dimyristoyl-phosphatidylcholine, dipentadecanoylphosphatidylcholine, dipalmitoylphosphatidyl-choline (DPPC) and distearoylphosphatidylcholine (DSPC); phosphatidylethanolamine esters such as dioleoylphosphatidylethanolamine and dipalmitoylphosphatidylethanol-amine (DPPE); phosphatidylserine; phosphatidylglycerol; phosphatidylinositol; etc. Phospholipids and other amphiphilic compounds can enhance stability of the present compositions.

Amphiphilic compounds can act as emulsifying agents in a composition of the invention. Any pharmaceutically acceptable emulsifying agent or combination thereof can be used, including without limitation medium and long chain monoglycerides and diglycerides, such as glyceryl monooleate, glyceryl monostearate, glyceryl monoisostearate and glyceryl monopalmitate, and polyglyceryl esters of fatty acids, such as polyglyceryl-3 oleate. Such agents can also function as emollients in the composition.

Factors affecting release rate of the estrogenic compound(s) can include the particular estrogenic compound(s) used, the physical form of the estrogenic compound(s) (e.g., whether in solution or in particulate form, and if particulate, average particle size), viscosity of the composition, selection and relative amounts of lipoidal compounds, including amphiphilic compounds, in the external phase, osmotic properties of the internal phase, and the relative volumes of the internal and external phases, among other factors. In a composition having the estrogenic compound(s) in the internal phase, and having a relatively small internal phase ratio (expressed as percentage of total volume occupied by the internal phase), the external phase tends to form a relatively thick membrane through which the estrogenic compound(s) must pass to be released; accordingly release rate can be significantly slowed in such a composition.

A suitable internal phase ratio can be established for any particular system by routine testing. In one embodiment the internal phase ratio is at least about 70% by volume.

Illustratively, a semi-solid composition of the invention such as a vaginal cream can have a viscosity of about 5,000 to about 1,000,000 centipoise, for example about 5,000 to about 750,000 centipoise, about 100,000 to about 800,000 centipoise, about 100,000 to about 400,000 centipoise, about 350,000 to about 750,000 centipoise, about 100,000 to about 550,000 centipoise, about 250,000 to about 400,000 centipoise, about 200,000 to about 350,000 centipoise, or about 350,000 to about 550,000 centipoise. Bioadherence of a composition to a mucosal surface requires, among other properties, sufficient viscosity to retain integrity of the composition. Optional ingredients that can increase viscosity, among other properties, include microcrystalline wax, colloidal silicon dioxide, and various pharmaceutically acceptable polymers including polysaccharides, cellulosic polymers such as carboxymethylcellulose, methylcellulose, hydroxypropylmethylcellulose, etc., polyethylene glycol, acrylate polymers and the like. In an illustrative embodiment, a composition useful herein is a thermally gelling formulation comprising a thermosetting polymer, e.g., a poloxamer such as poloxamer 407 (available, for example, as Pluronic™ F-127 of BASF).

In an illustrative embodiment, a vaginal cream comprises at least one estrogenic compound, for example estradiol, ethinyl estradiol or estrone, water, sorbitol, propylene glycol, at least one long chain monoglyceride, for example glyceryl monooleate, glyceryl monostearate, glyceryl monoisostearate or glyceryl monopalmitate, a chelating agent, for example edetate disodium, at least one antimicrobial preservative, for example methylparaben and/or propylparaben, mineral oil, microcrystalline wax and colloidal silicon dioxide, for example hydrophobically modified colloidal silicon dioxide.

A composition of the invention in the form of a vaginal cream can be prepared by known batch or continuous processes for preparing pharmaceutical creams. As in preparing conventional emulsions, shear force is applied to the components by use of a mixer, homogenizer, mill, impingement surface, ultrasound, shaking or vibration. Mixing shear should be at a relatively low level to prevent destruction of the emulsion by excess energy.

Illustratively, the internal and external phases are first prepared separately. In a typical batch process, the internal phase is added to the external phase while mixing in a planetary-type or other suitable mixer until a stable emulsion is formed. Addition rates and mixing speeds can be adjusted to optimize formation and viscosity of the emulsion. In a typical continuous process, the external phase is introduced into a continuous mixer that comprises a plurality of impellers, until it reaches the level of the lowest impeller in the mixing chamber. The two phases are then simultaneously introduced through the bottom of the mixer in proper proportion as the impellers rotate to apply shear to the components. The finished emulsion emerges through the top of the mixer. Flow rate through the mixing chamber and mixing speed can be adjusted to optimize formation and viscosity of the emulsion.

A composition of the invention can be administered topically to any part of the skin to deliver at least one estrogenic compound thereto for a local dermatological benefit, for example promotion of healing. Thus in one embodiment of the invention, a method for providing a local dermatological benefit to an area of skin comprises topically administering to the area of skin a composition comprising at least one estrogenic compound, and having at least one nonlipoidal internal phase and at least one lipoidal external phase that is bioadhesive to the skin surface, wherein the at least one estrogenic compound is present in an amount of about 5 to about 1000 μg estradiol equivalent per unit dose of the composition.

More particularly, a composition of the invention can be administered topically to external surfaces of the vulva and/or to surrounding areas of skin. In addition or alternatively, the composition can be administered intravaginally. In one embodiment, the composition is a vaginal cream, i.e., a semi-solid formulation adapted for administration to vaginal mucosal surfaces.

The amount of the composition to be administered will depend on the particular estrogenic compound or compounds present, the concentration of such compound or compounds in the composition, the frequency of administration (as determined for example by release rate) and other factors. As illustration, administration of 1 g of a composition containing 50 μg/g estradiol or an equivalent amount of an estrogenic compound other than estradiol, having a release period of 5 days, results in delivery of about 10 μg estradiol equivalent per day.

A vaginal cream of the invention can be administered to contact a mucosal surface in the vaginal cavity by means, for example, of an applicator that is optionally pre-filled with a single unit dosage amount of the cream. With the patient optionally in a supine position, the tip of the applicator can be gently inserted high in the vagina, for example in the posterior vaginal fornix, and the cream can be released through the tip by pushing on a plunger of the applicator.

A method comprising vaginal administration of a composition of the invention comprising about 5 to about 500 μg estradiol equivalent per unit dose is useful in treatment or prophylaxis of any hypoestrogenism-related condition local to the female urogenital system, in particular to the vaginal cavity and walls thereof, including associated surfaces of the vulva, cervix and urinary tract. Hypoestrogenism-related conditions for which such a method can be useful include without limitation lower urinary tract symptoms such as urinary incontinence (urge incontinence and stress incontinence), urgency and frequency of urination, nocturia, and dysuria; increased incidence of urinary tract infections; cervical dysplasia; and vulvodynia. More particularly, such a method is useful in treatment of atrophic vaginitis (objective signs of which include pallor, petechiae and/or friability of the vaginal mucosa) and conditions associated therewith, such as vulvovaginal dryness, irritation, pruritus, discharge and/or dyspareunia, especially in menopausal and postmenopausal patients. The method is generally useful in treatment of postmenopausal women with symptoms of urogenital aging.

A prolonged release period is enabled by compositions of some embodiments of the invention. Such a prolonged release period brings a number of benefits to the patient, including without limitation those discussed immediately below.

First, frequency of application can be significantly reduced by comparison with a composition having faster release. In general, frequency of application of a prolonged-release composition for effective treatment is once every 2 to 30 days, for example once or twice per month, once every 2 to 14 days, once every 2 to 10 days, or about 1 to about 3 times per week, illustratively about three times weekly, about twice weekly or about once weekly.

Second, the slow release from such a composition can result in maintenance of a therapeutically effective local concentration of estrogen without causing a major increase in systemic estrogen levels as measured, for example, by blood serum concentration. Risk of undesired or adverse side effects of increased serum estrogen level is thus minimized. This benefit is especially great for subpopulations of women for whom high levels of serum estrogen are believed to hold particular risk, such as breast cancer survivors.

Third, dosage amounts of the estrogenic compound can be reduced to levels close to the lowest effective dose for treatment of the local hypoestrogenism-related condition, for example atrophic vaginitis, taking advantage of the drug-sparing effect of slow release and minimizing adverse side effects.

In various embodiments of the method of the invention, the amount and release rate of the at least one estrogenic compound in a unit dose of the composition are selected to result, upon vaginal application as described above, in an increase in serum estradiol concentration of predominantly no more than about 50 pg/ml, predominantly no more than about 20 pg/ml, predominantly no more than about 10 pg/ml, predominantly no more than about 5 pg/ml or predominantly no more than about 2 pg/ml. The word “predominantly” in the present context means that during most (greater than 50%, typically greater than about 70%) of the release period following administration, serum estradiol does not exceed the stated concentration.

In other embodiments of the method of the invention, the amount and release rate of the at least one estrogenic compound in a unit dose of the composition are selected to result, upon vaginal application as described above, in a peak increase in serum estradiol concentration of no more than about 50 pg/ml, no more than about 20 pg/ml, no more than about 10 pg/ml, no more than about 5 pg/ml or no more than about 2 pg/ml.

Recently developed bioassays have made it possible to detect and quantify small changes in serum estradiol concentrations such as 20 pg/ml or less.

In one embodiment, a composition as described herein delivers an estrogenic compound in a dosage amount that is substantially less than is delivered by estrogen vaginal cream products on the market at the time of the present invention. An example of such a product is Estrace® estradiol vaginal cream of Warner Chilcott, containing 0.01% estradiol. The usual dosage range of Estrace® cream for treatment of vulvar and vaginal atrophy is 1 to 4 g daily, thereby delivering 100 to 400 μg estradiol per day. By comparison, a vaginal cream of the present embodiment illustratively delivers about 2 to about 50 μg, for example about 3 to about 30 μg, estradiol equivalent per day.

A frequently overlooked challenge in vaginal delivery of estrogen is that the vaginal mucosa and/or epithelium in patients with atrophic vaginitis absorbs estrogenic compounds such as estradiol more efficiently than the corresponding tissues of a healthy patient. Thus, as treatment of atrophic vaginitis by a method of the invention is effective in regenerating the vaginal mucosa and/or epithelium to a more healthy state, efficiency of absorption tends to go down.

Now provided is a method for treating a hypoestrogenism-related condition of the urogenital system of a female patient, the method comprising intravaginal administration of at least one estrogenic compound according to a treatment regimen wherein a series of compositions releasing a progressively increasing daily amount of the at least one estrogenic compound, expressed for example as estradiol equivalent, is administered over a period of at least about 1 month, for example about 1 to about 12 months.

The increase in daily amount of estradiol equivalent can be modulated to compensate for the reduced absorption resulting from progressive regeneration of the vaginal mucosa and/or epithelium. Depending on the severity of vulvovaginal atrophy, a starting dose of the at least one estrogenic compound can be one delivering about 2 to about 20 μg estradiol equivalent per day, a lower starting dose being appropriate in more severe cases and a higher starting dose in less severe cases. After an appropriate period, which will depend to some extent on the rate at which the vaginal mucosa and/or epithelium regenerates in response to the starting dose, but which typically can be about 1 to about 8 weeks, for example about 1 to about 4 weeks, the patient is transitioned to a higher dose, for example one delivering about 10 to about 50 μg estradiol equivalent per day. Further transitions to still higher doses can be included in the regimen if needed or desired, but typically a dosage delivering about 50 μg estradiol equivalent per day will not be exceeded. After a total period of typically at least about 3 months, for example about 3 to about 12 months, on such a regimen, occasionally longer if the patient is responding very slowly to the treatment, treatment can be terminated, or optionally continued indefinitely in the form of a maintenance dosage. A suitable maintenance dosage can deliver up to about 250 μg estradiol equivalent per day, but more typically delivers about 10 to about 25 μg estradiol equivalent per day.

The estrogenic compound(s) used according to such a regimen can vary over the course of the regimen, but typically the same compound or compounds are used throughout, only the dosage varying as described above.

The form of a composition used to deliver the estrogenic compound(s) according to a regimen as described above is not critical, and can include vaginal creams, thermally gelling formulations, tablets, pessaries and implants, e.g., vaginal rings. The composition, e.g., vaginal cream, can be one providing a release rate of the at least one estrogenic compound consistent with a once daily to once monthly, for example about once to about three times per week, dosing schedule.

In one embodiment the composition used at each stage in the regimen is a vaginal cream. The same cream can be used in successive stages, with increase in the amount of the cream administered; alternatively, the patient can be transitioned to a cream having a higher concentration of the at least one estrogenic compound and continue to administer the same amount. The cream can be one having conventional release properties, requiring daily application except for maintenance purposes. However, in a particular embodiment the composition is a slow-release cream, for example one having at least one nonlipoidal internal phase and at least one lipoidal external phase that is bioadhesive to a vaginal mucosal surface, wherein the at least one estrogenic compound is present in an amount of about 5 to about 500 μg estradiol equivalent per unit dose of the composition, and upon application of the composition to the vaginal mucosal surface the at least one estrogenic compound is released over a period of about 2 to about 30 days, for example about 2 to about 14 days or about 2 to about 10 days. The at least one estrogenic compound can be present in the nonlipoidal phase, the lipoidal phase or both; similar or different amounts can be present in the nonlipoidal and lipoidal phases.

Such a slow-release cream, for example one delivering a low dose of the at least one estrogenic compound (e.g., about 5 to about 50 μg estradiol equivalent per day) is well adapted for use according to the regimen described above.

For illustration only, during a starting period of about 2 weeks (the period from week 1 to week 2 of the treatment regimen), a composition releasing about 5 to about 10 μg, illustratively about 7 μg, estradiol equivalent per day can be administered. Such a composition can be one comprising, per unit dose, about 25 μg estradiol equivalent, for example in the form of ethinyl estradiol, administered twice weekly. Following this starting period, a composition releasing a greater amount of estrogen, for example about 10 to about 20 μg, illustratively about 14 μg, estradiol equivalent per day can be administered. From week 3 to week 12, for example, the composition administered can be one comprising about 50 μg estradiol equivalent per unit dose, administered twice weekly; later, for example from week 13 to week 26, a composition comprising about 100 μg estradiol equivalent per unit dose can be administered twice weekly. After week 26, a maintenance dose can be administered, for example about 150 μg once weekly.

There is further provided a kit for use according to the present method. The kit comprises a plurality of vaginal creams, each having at least one nonlipoidal internal phase and at least one lipoidal external phase that is bioadhesive to a vaginal mucosal surface, wherein the at least one estrogenic compound is present in an amount of about 5 to about 500 μg estradiol equivalent per unit dose of the cream, and upon application of the cream to the vaginal mucosal surface the at least one estrogenic compound is released over a period of about 3 hours to about 30 days, for example about 3 hours to about 14 days, about 3 hours to about 10 days, or about 2 to about 10 days. The plurality of vaginal creams are adapted, when applied in progressive sequence over a period of at least about 1 month, for example about 1 to about 12 months, to the vaginal mucosal surface, to release a progressively increasing daily amount of the at least one estrogenic compound. The kit optionally further comprises instructions, in hard-copy and/or electronic form, for administration according to a prescribed regimen.

### EXAMPLES

The following examples are merely illustrative, and do not limit this disclosure in any way.

Each of the compositions detailed below can be prepared by any method known in the art for preparing semi-solid emulsions, including batch and continuous processes as described hereinabove.

### Example 1

Estradiol Cream

| Ingredient | % w/w |
|---|---|
| water, purified, USP | 39.817 |
| sorbitol solution, USP | 39.978 |
| propylene glycol, USP | 5.000 |
| edetate disodium, USP | 0.050 |
| estradiol, USP | 0.002 |
| mineral oil, USP | 8.032 |
| polyglyceryl-3-oleate | 2.713 |
| glyceryl monoisostearate | 2.713 |
| microcrystalline wax, NF | 0.452 |
| silicon dioxide, hydrophobic | 1.013 |
| methylparaben, NF | 0.180 |
| propylparaben, NF | 0.050 |
| Total | 100.000 |

### Example 2

Ethinyl Estradiol Cream

| Ingredient | % w/w |
|---|---|
| water, purified, USP | 43.320 |
| sorbitol solution, USP | 39.996 |
| edetate disodium, USP | 0.050 |
| ethinyl estradiol, USP | 0.004 |
| mineral oil, USP | 10.000 |
| PEG 30 dipolyhydroxystearate | 4.000 |
| glyceryl monoisostearate | 2.000 |
| microcrystalline wax, NF | 0.400 |
| methylparaben, NF | 0.180 |
| propylparaben, NF | 0.050 |
| Total | 100.000 |

### Example 3

Estrone Cream

| Ingredient | % w/w |
|---|---|
| water, purified, USP | 45.292 |
| sorbitol solution, USP | 39.598 |
| edetate disodium, USP | 0.050 |
| estrone, USP | 0.010 |
| mineral oil, USP | 7.000 |
| polyglyceryl-3-oleate | 2.700 |
| glyceryl monoisostearate | 2.700 |
| lecithin | 1.000 |
| microcrystalline wax, NF | 0.400 |
| silicon dioxide, hydrophobic | 1.000 |
| methylparaben, NF | 0.200 |
| propylparaben, NF | 0.050 |
| Total | 100.000 |

### Example 4

Estradiol Vaginal Cream Formulations

| Ingredient | Wt % | | | | |
|---|---|---|---|---|---|
| Estradiol (target μg/g): | 10 | 30 | 50 | 70 | 100 |
| water, purified, USP | 39.819 | 39.817 | 39.815 | 39.813 | 39.81 |
| sorbitol solution, USP | 40.000 | 40.000 | 40.000 | 40.000 | 40.00 |
| propylene glycol, USP | 5.000 | 5.000 | 5.000 | 5.000 | 5.00 |
| edetate disodium, USP | 0.050 | 0.050 | 0.050 | 0.050 | 0.05 |
| estradiol, USP | 0.001 | 0.003 | 0.005 | 0.007 | 0.01 |
| mineral oil, USP | 8.000 | 8.000 | 8.000 | 8.000 | 8.00 |
| polyglyceryl-3-oleate | 2.750 | 2.750 | 2.750 | 2.750 | 2.75 |
| glyceryl monoisostearate | 2.750 | 2.750 | 2.750 | 2.750 | 2.75 |
| microcrystalline wax, NF | 0.400 | 0.400 | 0.400 | 0.400 | 0.40 |
| silicon dioxide, | 1.000 | 1.000 | 1.000 | 1.000 | 1.00 |
| hydrophobic | | | | | |
| methylparaben, NF | 0.180 | 0.180 | 0.180 | 0.180 | 0.18 |
| propylparaben, NF | 0.050 | 0.050 | 0.050 | 0.050 | 0.05 |

Compositions of Examples 1-4 can be administered in a dosage amount of about 5 g to a vulvovaginal surface, more particularly a vaginal mucosal surface, for treatment of a hypoestrogenism-related condition of the urogenital system of a female subject, for example atrophic vaginitis, according to a method as described herein.

All patents and publications cited herein are incorporated by reference into this application in their entirety.

The words “comprise”, “comprises”, and “comprising” are to be interpreted inclusively rather than exclusively.

What is claimed is:

1. A pharmaceutical composition comprising at least one estrogenic compound, the composition being adapted for application in a unit dose amount to a vulvovaginal surface and having at least one nonlipoidal internal phase and at least one lipoidal external phase that is bioadhesive to the vulvovaginal surface, wherein the at least one estrogenic compound is present in an amount of about 5 to about 1000 μg estradiol equivalent per unit dose of the composition, and upon application of the composition to the vulvovaginal surface the at least one estrogenic compound is released over a period of about 3 hours to about 30 days.

2. The composition of claim 1, wherein the vulvovaginal surface to which the composition is adapted for application is a vaginal mucosal surface.

3. The composition of claim 2, wherein the at least one estrogenic compound is present in an amount of about 5 to about 500 μg estradiol equivalent per unit dose of the composition, and upon application of the composition to the vaginal mucosal surface, the at least one estrogenic compound is released over a period of about 2 to about 10 days.

4. The composition of claim 2 that is in a form of a vaginal cream.

5. The composition of claim 1, wherein the at least one estrogenic compound is a steroid.

6. The composition of claim 1, wherein the at least one estrogenic compound is selected from the group consisting of conjugated estrogenic hormones, estradiol, ethinyl estradiol, estriol and estrone.

7. The composition of claim 1, wherein the at least one estrogenic compound is estradiol or ethinyl estradiol.

8. The composition of claim 1, wherein the at least one estrogenic compound is present in an amount of about 25 to about 250 μg estradiol equivalent per unit dose of the composition.

9. The composition of claim 1, wherein the at least one estrogenic compound is released over a period consistent with a once daily to once monthly dosing schedule.

10. The composition of claim 1, wherein the at least one estrogenic compound is released over a period consistent with a once to three times per week dosing schedule.

11. A vaginal estrogen delivery system comprising the composition of claim 4 and an applicator.

12. The delivery system of claim 11, wherein the applicator is disposable.

13. The delivery system of claim 12, wherein the applicator is prefilled with a unit dose amount of the composition.

14. A method for providing a local dermatological benefit to an area of skin, the method comprising topically administering to the area of skin a composition comprising at least one estrogenic compound, and having at least one nonlipoidal internal phase and at least one lipoidal external phase that is bioadhesive to the skin surface, wherein the at least one estrogenic compound is present in an amount of about 5 to about 1000 μg estradiol equivalent per unit dose of the composition.

15. A method for treating a hypoestrogenism-related condition of the urogenital system of a female patient, the method comprising administering to a vulvovaginal surface a pharmaceutical composition that comprises at least one estrogenic compound, the composition having at least one nonlipoidal internal phase and at least one lipoidal external phase that is bioadhesive to the vulvovaginal surface, wherein the at least one estrogenic compound is present in an amount of about 5 to about 500 μg estradiol equivalent per unit dose of the composition, and upon application of the composition to the vulvovaginal surface the at least one estrogenic compound is released over a period of about 3 hours to about 30 days.

16. The method of claim 15, wherein the vulvovaginal surface to which the composition is administered is a vaginal mucosal surface.

17. The method of claim 15, wherein the condition is selected from the group consisting of urinary incontinence, urgency and frequency of urination, nocturia, dysuria, increased incidence of urinary infections, cervical dysplasia, vulvodynia, atrophic vaginitis, vulvovaginal dryness, irritation, pruritus, discharge, dyspareunia and postmenopausal urogenital aging.

18. The method of claim 15, wherein the condition is atrophic vaginitis or a disorder associated therewith.

19. The method of claim 15, wherein the composition is administered at a frequency of about 1 to about 3 times per week.

20. The method of claim 15, wherein the amount and release rate of the at least one estrogenic compound are selected to result in predominantly no more than a 50 pg/ml increase in serum estradiol concentration.

21. The method of claim 15, wherein the amount and release rate of the at least one estrogenic compound are selected to result in predominantly no more than a 10 pg/ml increase in serum estradiol concentration.

22. The method of claim 15, wherein the amount and release rate of the at least one estrogenic compound are selected to result in predominantly no more than a 5 pg/ml increase in serum estradiol concentration.

23. The method of claim 15, wherein the amount and release rate of the at least one estrogenic compound are selected to result in predominantly no more than a 2 pg/ml increase in serum estradiol concentration.

24. A method for treating a hypoestrogenism-related condition of the urogenital system of a female patient, the method comprising intravaginal administration of at least one estrogenic compound according to a treatment regimen wherein a series of compositions releasing a progressively increasing daily amount of the at least one estrogenic compound is administered over a period of at least about 1 month.

25. The method of claim 24, wherein the increase in daily amount of the at least one estrogenic compound is modulated to compensate for reduced absorption resulting from progressive regeneration of the vaginal mucosa and/or epithelium.

26. The method of claim 24, wherein the regimen comprises a starting dose of the at least one estrogenic compound effective to deliver about 2 to about 20 μg estradiol equivalent per day for about 1 to about 8 weeks, followed by transition to a higher dose of the at least one estrogenic compound effective to deliver about 10 to about 50 μg estradiol equivalent per day.

27. The method of claim 24, wherein the regimen comprises further transition to a still higher dose of the at least one estrogenic compound, not exceeding a dose effective to deliver about 50 μg estradiol equivalent per day.

28. The method of claim 24, wherein the regimen is continued for a total period of at least about 3 months.

29. The method of claim 24, wherein the regimen is continued for a total period of about 3 to about 12 months.

30. The method of claim 29, wherein at the conclusion of said period the regimen is continued indefinitely in the form of a maintenance dosage.

31. The method of claim 24, wherein the at least one estrogenic compound is a steroid.

32. The method of claim 24, wherein the at least one estrogenic compound is selected from the group consisting of conjugated estrogenic hormones, estradiol, ethinyl estradiol, estriol and estrone.

33. The method of claim 24, wherein the at least one estrogenic compound is estradiol or ethinyl estradiol.

34. The method of claim 24, wherein the compositions are independently selected from the group consisting of vaginal creams, thermally gelling formulations, tablets, pessaries and implants.

35. The method of claim 24, wherein the composition used at each stage in the regimen is a vaginal cream.

36. The method of claim 35, wherein the at least one estrogenic compound is present in an amount of about 25 to about 250 μg estradiol equivalent per unit dose of the vaginal cream.

37. The method of claim 35, wherein the at least one estrogenic compound is released over a period consistent with a once daily to once monthly dosing schedule.

38. The method of claim 35, wherein the at least one estrogenic compound is released over a period consistent with a once to three times per week dosing schedule.

39. The method of claim 35, wherein the vaginal cream has at least one nonlipoidal internal phase and at least one lipoidal external phase that is bioadhesive to a vaginal mucosal surface, wherein the at least one estrogenic compound is present in an amount of about 5 to about 500 μg estradiol equivalent per unit dose of the vaginal cream, and wherein upon application of the composition to the vaginal mucosal surface the at least one estrogenic compound is released over a period of about 2 to about 30 days.

40. The method of claim 35, wherein the vaginal cream is administered with the aid of an applicator.

41. The method of claim 40, wherein the applicator is disposable.

42. The method of claim 41, wherein the applicator is prefilled with a unit dose amount of the vaginal cream.

43. A kit comprising a plurality of vaginal creams comprising at least one estrogenic compound, wherein
(a) each vaginal cream has at least one nonlipoidal internal phase and at least one lipoidal external phase that is bioadhesive to a vaginal mucosal surface, the at least one estrogenic compound being present in each cream in an amount of about 5 to about 500 μg estradiol equivalent per unit dose of the cream, the at least one estrogenic compound, upon application of the cream to the vaginal mucosal surface, being released over a period of about 3 hours to about 30 days;
(b) the plurality of vaginal creams are adapted, when applied in progressive sequence over a period of at least about 1 month to the vaginal mucosal surface, to release a progressively increasing daily amount of the at least one estrogenic compound.

44. The kit of claim 43, further comprising instructions for administration according to a prescribed regimen.
